(19) United States
(12) Patent Application Publication
(10) Pub. No.: US 2004/0088005 A1
(43) Pub. Date: May 6, 2004

(54) Pacifier

(76) Inventor: Joseph J. Boyd, Cumming, GA (US)

Correspondence Address: TROUTMAN SANDERS LLP, BANK OF AMERICA PLAZA, SUITE 5200, 600 PEACHTREE STREET , NE, ATLANTA, GA, 30308-2216, US

(21) Appl. No.: 10/287,365

(22) Filed: Nov. 4, 2002

(51) Int. Cl. A61B 17/06; A61J 17/00

(52) U.S. Cl. 606/234

(57) ABSTRACT

A novel nipple protection device designed to prevent the nipple of a pacifier or bottle, used by a mammal, from contacting the ground when dropped is disclosed. The basic form of the invention has fixed guards, on each side of a nipple, attached to a housing, which protrude beyond the nipple and prevent it from touching the floor if it falls. The guard members can be rigid or flexible. In another embodiment, the guard members are hinged and light springs are used to partly close them against the mammal's cheeks when in use or around the nipple when the pacifier or bottle falls. A retractable nipple may also be included with either the fixed or pivotal guard designs to further protect the nipple from contamination when dropped. Finally, another variation is to replace the nipple with a straw and the bottle with any other container.

### BACKGROUND OF THE INVENTION

1. Field of the Invention

The present invention relates generally to pacifiers and nursing bottles, and more specifically to a novel nipple protection device designed to prevent contamination of the nipple of a pacifier or bottle if the pacifier or bottle falls to the floor.

2. Description of the Related Art

Presently, baby pacifiers have a large base or housing that prevents the baby from swallowing or choking on them. Such pacifiers, however, lack sanitary provisions to prevent the nipple from contact with the floor when the pacifier is dropped. This is also true of baby bottles. It is not uncommon to see a baby drop his pacifier or bottle, or even throw it to the ground just to see if his mother will pick it up. Frequently, the mother simply picks up the pacifier or bottle, wipes off the nipple with her hand, and then places it back into the baby's mouth. Even if the mother's hands are clean, some of the contaminants from the floor remain on the nipple.

Nor is wiping the nipple off with a cloth sufficient either, as it is likely to have been contaminated with bacteria and other micro-organisms, requiring a thorough cleaning. If the mother is out shopping or otherwise away from home, such a cleaning may be very inconvenient, if not impossible. Yet if the nipple is placed back into the baby's mouth without being cleaned, there is a strong likelihood of the baby becoming sick or infected. Preventing the nipple from becoming contaminated in the first place is thus preferable, rather than having to clean or sanitize the nipple after it has been dropped.

Conventional baby pacifiers and bottles do not prevent the nipple from touching the floor when dropped. What is needed then is a sanitary nipple guard that will keep the nipple off the floor and greatly reduce infant infections. It is to such a device that that the present invention is primarily directed.

### BRIEF SUMMARY OF THE INVENTION

The present invention includes fixed, elongated guard members projecting on sides of a suckling device that protrude beyond the suckling device, preventing it from touching the floor if the pacifier falls. The guard members can also be flexible so that they can bend even closer toward the suckling device or against the baby's cheeks while in use. The term suckling device as used herein will be understood to mean any device capable of being sucked on by a mammal, including but not limited to a nipple on a pacifier, a nipple on a bottle, a teething ring, and a straw.

Briefly described, in a preferred form for a pacifier, the present invention is a standard pacifier-type nipple, with a housing that supports the nipple, having pivotal guard members on the housing that at least partially close around the nipple when the nipple is not in the baby's mouth. The guard members additionally include tabs that facilitate opening the guard members from around the nipple. When the tabs on the guard members are squeezed together, the guard members open flat and allow the nipple to be placed into the baby's mouth. Light springs are used to close the guard members lightly against the baby's cheeks while it has the nipple in its mouth. If the pacifier is dropped, the guard members will then return to the closed position around the nipple. Only enough spring force to close the guard members when not in use is required to prevent the nipple of the pacifier from touching the floor when it falls.

In another embodiment, the nipple may also be retractable into the housing. The nipple is held at least partially inside the housing by a retraction member such as a light spring or the elasticity of a support diaphragm. When the nipple is placed in the baby's mouth, the baby sucks on the nipple and the nipple is pulled out to the full position. If the baby releases the nipple, the nipple is retracted into the housing by the retraction member, and if the pacifier falls or is dropped, the nipple thus does not touch the floor due to the retraction of the nipple.

This same sanitary-type nipple may be applied to the cap of a bottle or container. When used this way, a preferred embodiment includes the housing with accompanying guard members fastened to a bottle top instead of using a standard baby bottle cap. As with the pacifier embodiments, the guard members can be either fixed on the housing or pivotally attached. This design may also be combined with the retractable nipple, so that the nipple is protected from touching the floor or ground when it is dropped by both the guard members as well as the retracting feature of the nipple. Finally, the nipple may be replaced by a straw as the baby grows to facilitate adjustment from nipples to normal sipper cups.

A principle object of the present invention is to provide an innovative nipple protection device designed to prevent the nipple of a pacifier or baby bottle from coming in contact with the ground or floor when it falls or is dropped.

It is another object of the present invention to provide a nipple protection device that will reduce the need for cleaning and sterilizing the nipple of a pacifier or baby bottle after the pacifier or baby bottle comes in contact with the ground.

Yet another object of the present invention is providing a convenient alternative to standard pacifiers and baby bottle nipples that is economical, sanitary, and easy to use.

Still another object of the present invention is to provide a sanitary device that will facilitate weaning a baby from its mother.

These and other objects, features and advantages of the present invention will become more apparent upon reading the following specification in conjunction with the accompanying drawings.

### BRIEF DESCRIPTION OF THE FIGURES

FIG. 1 is a front view of a preferred embodiment of the present invention.

FIG. 2 is a front view of the preferred embodiment in FIG. 1 adapted to fit onto a baby bottle.

FIG. 3 is a front view showing another embodiment of the present invention adapted to fit a baby bottle and having a retractable nipple.

FIG. 4 is a front view of the embodiment in FIG. 3 showing the retractable nipple in the extended position.

FIG. 5 is a front view of the embodiment in FIG. 3 showing the retractable nipple in the retracted position.

FIG. 6 is a front view of yet another embodiment of the present invention with the guard members in the open position.

FIG. 7 is a front view of the embodiment in FIG. 6 with the guard members in the closed position.

FIG. 8 is a side view of the embodiment in FIG. 7 showing the guard members keeping the nipple clear of the floor.

FIG. 9 is a bottom view of FIG. 8 showing the guard members and closing member.

### DETAILED DESCRIPTION OF THE PREFERRED EMBODIMENTS

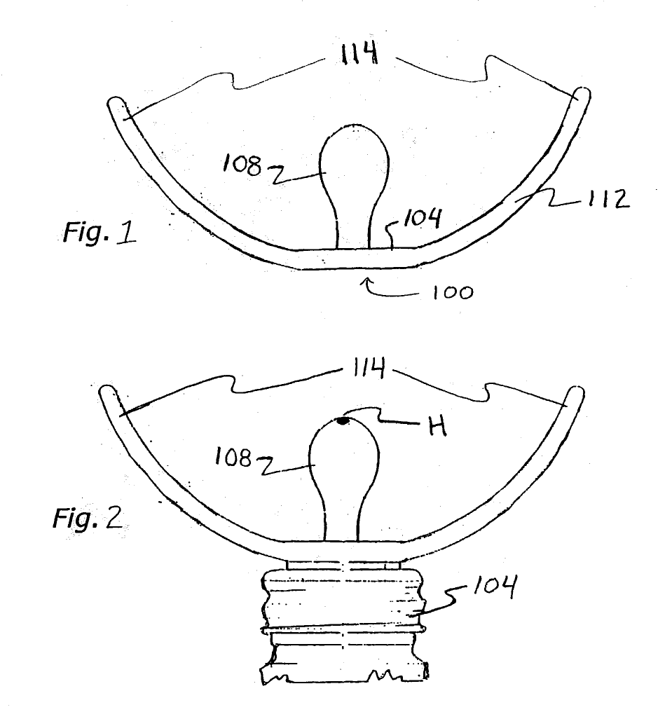

Referring now in detail to the drawing figures, wherein like reference numerals represent like parts throughout the several views, FIG. 1 illustrates a front view of a preferred embodiment of the present invention. The invention is a pacifier 100 comprising a housing 104 with a suckling device 108 attached and a guard 112 extending outward and upward from the housing 104 beyond the suckling device 108, so in the event the pacifier 100 is dropped, the suckling device 108 is protected from touching the floor. In a preferred embodiment, suckling device 108 is a standard pacifier-type nipple, and the guard 112 is fixed, elongated “wing-like” guard members 114, projecting on each side of the nipple 108, that are long enough to protrude beyond the height of the nipple 108, protecting it from touching the floor if the pacifier falls. The guard members 114 may also be flexible, so that they can bend even closer toward the nipple 108 or against a baby's cheeks while in use.

FIG. 2 is a front view of the embodiment in FIG. 1 adapted to fit onto a baby bottle. Housing 104 is adapted to screw on the top of a baby bottle. In addition, nipple 108 has been adapted with a hole H through which a baby may drink from the bottle. A person of ordinary skill in the art, however, will recognize that the present invention is adaptable to any beverage container and is therefore not limited to baby bottles. Finally, the guard members 114 are shown extending beyond the height of the nipple 108, protecting it from contamination if the bottle is dropped.

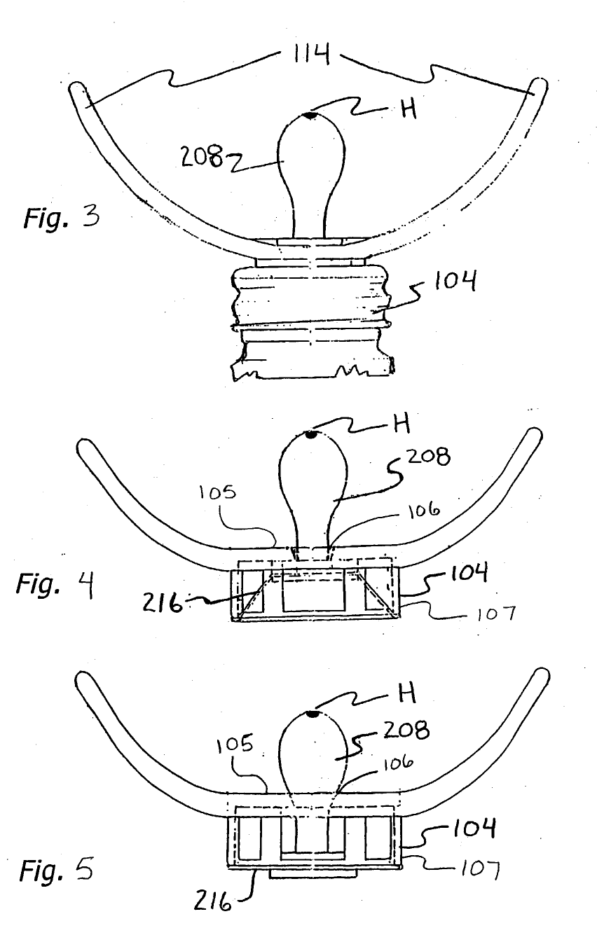

Another embodiment of the present invention adapted to fit a baby bottle incorporates a retractable nipple as shown in FIG. 3. Housing 104 is adapted to fit onto a baby bottle and nipple 208 is retractable into housing 104. Thus, nipple 208 is protected from touching the ground if the bottle is dropped by both the guard members 114 as well as the ability to retract into housing 104.

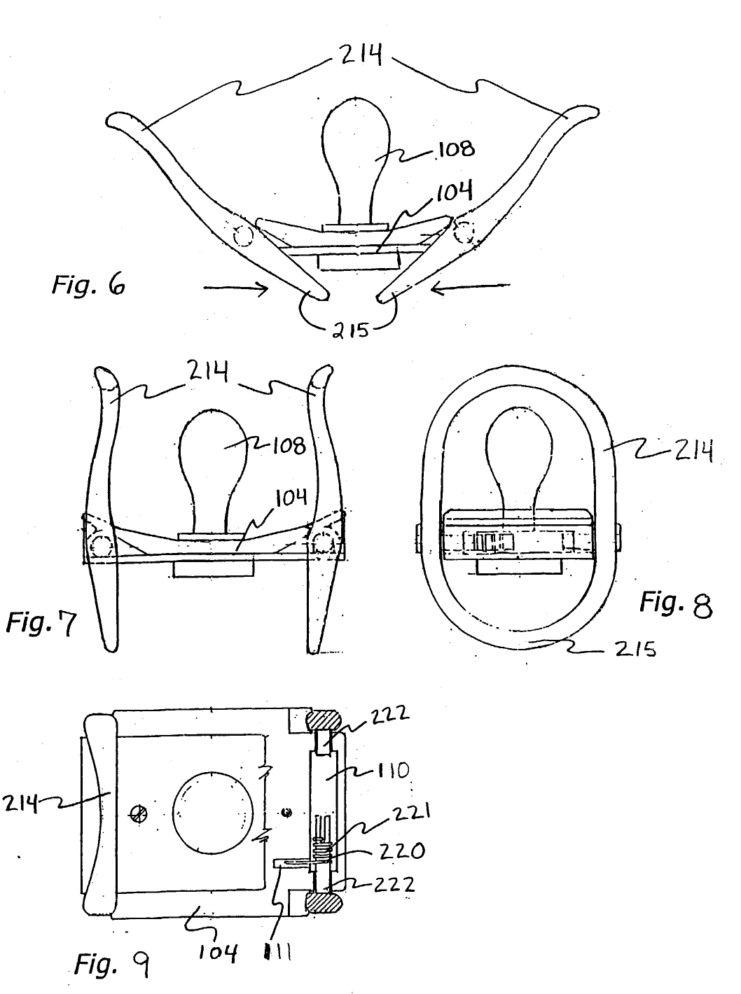

FIGS. 4 and 5 show two different positions of the retractable nipple type assembly. FIGS. 4 and 5 also show an embodiment for the construction of the housing 104. Housing 104 has a top wall 105 with an aperture 106 fitted around nipple 208, and at least one side wall 107 depending from the top wall 105, forming an opening to fit onto a baby bottle. FIG. 4 is a front view of the embodiment in FIG. 3 showing the retractable nipple in an extended position. Nipple 208 is connected to the inside of housing 104 by a retraction member 216 and is shown in the extended position in FIG. 4. FIG. 5 is a front view of the same embodiment in FIG. 3 showing the retractable nipple in a retracted position. Nipple 208 is at least partially within the housing 104 via retraction member 216 such as a light spring or the elasticity of a rubber support diaphragm.

FIG. 6 is a front view of yet another embodiment of the present invention with the guard members in an open position. In this embodiment, two guard members 214 are pivotally attached to housing 104. The nipple 108 communicates with the housing 104 that supports the two guard members 214 which pivot about the housing 104. The guard members 214 are shown in an open position to allow the nipple 108 to be placed into a baby's mouth. The guard members are opened by pressing tabs 215, which may or may not be part of the guard members 214.

FIG. 7 is a front view of the embodiment in FIG. 6 with the guard members in a closed position. In FIG. 7, the two guard members 214 are in the closed position, partially around the nipple 108 such as when the pacifier is removed from the baby's mouth. Light pressure from a closing member 220 (FIG. 9) closes the guard members 214 around nipple 108 which prevents it from touching the floor when the pacifier falls.

A side view of the embodiment in FIG. 7 is shown in FIG. 8 demonstrating how the guard members keep the nipple clear of the floor. In FIG. 8, the guard members 214 are oval shaped at the top and at the bottom where they form tabs 215.

FIG. 9 is a bottom view of FIG. 8 showing the guard members and closing member 220. A section of the housing 104 has been removed to show an embodiment of an assembly for the closing member 220. As shown, the closing member 220 includes a spring assembly that will positively, but lightly, close the guard members 214 when not in use. The spring 221 is shown coiled around a hinge or a pin 222 that is used to attach each guard member 214 to the housing 104. As illustrated, the hinge pins 222 rest in a socket 110 of the housing 104, and the spring 221 is coiled around the inside extension of the hinge pin 222. One end of the spring 221 is fastened to the extension of the hinge pin 222 and the other end rests in a slot 111 in the housing 104. A person of ordinary skill in the art, however, will recognize that many other methods of pivoting the guard members and providing a closing member may be used without changing the scope of the invention. In addition, this embodiment may also include the retractable nipple feature by including a retraction member such as that shown in FIGS. 3-5. Moreover, the housing 104 on this embodiment may be adapted to fit onto a container to deliver the contents of the container to a standard baby bottle nipple. Finally, the embodiments described herein may also be used for keeping a suckling device, such as a straw of a drink bottle, clean and are thus not limited to a pacifier or baby bottle.

The suckling device of the present invention is preferably constructed of a pliant or elastomeric material such as a natural or synthetic rubber, latex, or a softer plastic material, whereas, the housing of the present invention and accompanying guard members are preferably constructed of a more rigid material such as plastic, metal, or various other polymers. In addition, the rigid material can be overlaid with a softer material, or vice versa. A unitary molded structure is preferable for the embodiment having fixed guard members with a bendable material incorporated into the tips of the guard members so that they may be manipulated and will hold a shape. Finally, an antibacterial coating may also be applied to any part of the present invention to increase sanitation.

Numerous characteristics and advantages have been set forth in the foregoing description, together with details of structure and function. While the invention has been disclosed in its preferred form, it will be apparent to those skilled in the art that many modifications, additions, and deletions, especially in matters of shape, size, and arrangement of parts, can be made therein without departing from the spirit and scope of the invention and its equivalents as set forth in the following claims. Therefore, other modifications or embodiments as may be suggested by the teachings herein are particularly reserved as they fall within the breadth and scope of the claims here appended.

What is claimed is:

1. A pacifier protectable from the ground, the pacifier comprising:
a housing;
a suckling device having a tip, the suckling device in communication with the housing; and
a guard in communication with the housing, the guard extending from the housing beyond the tip of the suckling device, such that if the pacifier contacts the ground, the guard prevents the tip of the suckling device from contact with the ground.

2. The pacifier of claim 1, the guard comprising:
at least two guard members each having a tip and a base, the tips of the guard members extending upwardly from the housing and beyond the tip of the suckling device.

3. The pacifier of claim 2, wherein the guard members are positioned opposite one another on the housing with the suckling device positioned therebetween.

4. The pacifier of claim 3, wherein each guard member is attached to the housing at the base of each guard member, and the tips of each guard member extend outwardly and upwardly from the housing and beyond the tip of the suckling device.

5. The pacifier of claim 4, wherein the suckling device is a nipple having a hole therethrough, and the housing is adapted to communicate with a container and deliver the contents of the container to the nipple.

6. The pacifier of claim 5, wherein the tips of each guard member are flexible and may be bent to conform to a user's face

7. The pacifier of claim 6, wherein the nipple is a straw.

8. The pacifier of claim 3, wherein each guard member is pivotally attached to the housing, such that the tips of each guard member may be moved between a closed position proximate to the suckling device and an open position distal to the suckling device.

9. The pacifier of claim 8, wherein each guard member is pivotally attached to the housing between the tip and the base of each guard member.

10. The pacifier of claim 9, wherein the pacifier further comprises:
a closing member to move the tips of the guard members toward the closed position when not in use, such that when the base of the guard members are squeezed together, the tips of the guard members move from the closed position toward the open position, and if the base of the guard members are released, the tips of the guard members will return toward the closed position proximate to the suckling device and come to rest on the user's face when in use, or return fully to the closed position if released by the user, preventing the tip of the suckling device from coming in contact with the ground.

11. The pacifier of claim 10, wherein the closing member is at least one spring.

12. The pacifier of claim 11, wherein the suckling device is a nipple having a hole therethrough and the housing is adapted to communicate with a beverage container and deliver the contents of the container to the nipple.

13. The pacifier of claim 12, wherein the nipple is a straw.

14. A pacifier protectable from the ground, the pacifier comprising:
a housing;
a suckling device having a tip, the suckling device in communication with the housing;
a guard in communication with the housing, the guard extending upwardly from the housing toward the tip of the suckling device; and
at least one retraction member for retracting the suckling device into the housing, such that if the suckling device is released by the user, the suckling device will be retracted into the housing bringing the tip of the suckling device below the guard to prevent the tip of the suckling device from contact with the ground.

15. The pacifier of claim 14, the guard comprising:
at least two guard members each having a tip and a base, the tips of the guard members extending upwardly from the housing toward the tip of the suckling device.

16. The pacifier of claim 15, wherein the suckling device is a nipple having a hole therethrough and the housing is adapted to communicate with a container and deliver the contents of the container to the nipple.

17. The pacifier of claim 16, wherein the housing comprises:
a top wall with an aperture for communicating with the nipple; and
at least one side wall, the side wall depending from the top wall and defining an open end in the housing, the open end adapted to communicate with a beverage container.

18. The pacifier of claim 17, wherein the nipple is a straw.

19. The pacifier of claim 15, wherein each guard member is pivotally attached to the housing, such that the tips of each guard member may be moved between a closed position proximate to the suckling device and an open position distal to the suckling device, the pacifier further comprising a closing member to move the tips of the guard members toward the closed position when not in use, wherein the suckling device is a nipple having a hole therethrough and the housing is adapted to communicate with a container and deliver the contents of the container to the nipple.

20. A pacifier protectable from the ground, the pacifier comprising:
a suckling device having a hole therethrough;
a housing in communication with the suckling device, the housing adapted to communicate with a container and deliver the contents of the container to the suckling device;
at least one retraction member for retracting the suckling device into the housing;
at least two pivotal guard members having a base and a tip, the guard members in communication with the housing, such that the tips of each guard member may be moved between a closed position proximate to the suckling device and an open position distal to the suckling device, the guard members each having a tab at its base for opening the guard members; and
a closing member to move the tips of the guard members toward the closed position when the tabs are released or when the suckling device is released by the user, the closing member being in communication with the guard members and the housing, such that if the user releases the suckling device, the suckling device will retract into the housing and the guard members will move toward the closed position protecting the suckling device from the ground.
